(19) United States
(12) Patent Application Publication
(10) Pub. No.: US 2013/0269712 A1
(43) Pub. Date: Oct. 17, 2013

(54) ADJUSTABLE BITE PLANE APPLIANCE

(71) Applicant: Douglas Awde, London (CA)

(72) Inventor: Douglas Awde, London (CA)

(21) Appl. No.: 13/863,822

(22) Filed: Apr. 16, 2013

(30) Foreign Application Priority Data
Apr. 16, 2012 (CA) 2774962

(51) Int. Cl. A61F 5/56

(52) U.S. Cl. CPC A61F 5/566
USPC 128/848

(57) ABSTRACT

A bite plane apparatus for applying to a patient's teeth is provided. The bite plane apparatus has an adjustable portion with a moveable member in a manibular portion that corresponds with a raised portion on a maxillary portion to dispose the patient's jaw anteriorly. A bite plane having adjustable wedges in the mandibular portion to increase or decrease the distance between the occlusal surfaces of the mandibular and maxillary teeth. The adjustable bite planes have a left and right occusal portions connected with a bridge.

### RELATED APPLICATIONS

This application claims priority to Canadian Patent Application No. 2,774,962, filed Apr. 16, 2012, which is incorporated in its entirety herein by reference.

### BACKGROUND

1. Field of the Invention

This invention relates to removable oral appliances. More specifically, this invention relates to removable bite plane and snoring appliances.

2. Background of the Invention

A conventional bite plane appliance generally conforms to and fits over a patient's teeth, and has one acrylic occlusal plane that covers the occlusal surface of the teeth so that the maxillary and mandibular teeth cannot be brought together into contact. Posterior bite plane appliances are typically used to retain or dispose the patient's mandible in a desired position to prevent indications such as grinding and clenching of the teeth and snoring.

To prevent and treat such problems, proper occlusion is critical. According to the prior art, maximum effectiveness is achieved by using bite planes having custom-thicknesses as determined by trial and error. This requires that the patient attend at the dental professional's office for an initial fitting, and thereafter attend again for customization of the bite planes. This can often be an iterative procedure, requiring that the patient take the appliance away after modification of the bite plane to determine if it is comfortable, resolves the pathology for which the appliance was installed, and does not create other problems; and if not, reattend at the professional's office for further customization of the bite planes.

Such personalized fitting of conventional bite plane appliances tends to be difficult, typically requiring the addition of layers of acrylic to increase the thickness of the bite planes in small increments each followed by an interval of observation to determine if further modification is necessary. Thus, properly fitting a bite plane appliance can be expensive and time consuming for the patient, often requiring attendance at the dental practitioner's office multiple times for adjustments.

Additionally, where removable occlusal shims or any other removable parts are attached to a bite plane base to treat a patient's pathology, they must be affixed to the bite plane base in a precise and immovable position in order to effectively address the patient's pathology. Where the removable parts are easily dislodged, treatment can be defeated and, at worst, can be dangerous to the patient.

Adjustable bite planes that can be easily modified and customized without removable parts are therefore desirable.

### BRIEF DESCRIPTION OF THE DRAWINGS

In drawings which illustrate by way of example only a preferred embodiment of the invention,

FIG. 1 is an exploded perspective view of an adjustable mandibular bite plane appliance and a cooperating maxillary bite plane appliance comprising removable shims;

FIG. 2 is a perspective view of the adjustable mandibular bite plane appliance of FIG. 1 shown with an adjustment key operating thereon to advance an abutting surface;

FIG. 3 is a perspective view of the adjustable mandibular bite plane appliance and cooperating maxillary bite plane appliance of FIG. 1 held together by elastic bands;

FIG. 4 is a left side view of the adjustable mandibular bite plane appliance and cooperating maxillary bite plane appliance of FIG. 1 forcing a slightly anterior disposition of the mandible when the mouth is closed;

FIG. 5 is a left side view of the adjustable mandibular bite plane appliance and cooperating maxillary bite plane appliance of FIG. 1 forcing a greater anterior disposition of the mandible when the mouth is closed;

FIG. 6 is a cranial perspective view of an adjustable maxillary bite plane appliance;

FIG. 7 is a caudal perspective view of the adjustable maxillary bite plane appliance of FIG. 6;

FIG. 8 is a caudal plan view of the right wedge portion of the adjustable maxillary bite plane appliance of FIG. 6 where the wedge is in a retracted position;

FIG. 9 is a caudal plan view of the right wedge portion of the adjustable mandibular bite plane appliance of FIG. 6 where the wedge is in an expanded position;

FIG. 10 is a buccal side view of the left wedge portion of the adjustable maxillary bite plane appliance of FIG. 6 where the wedge is in a retracted position;

FIG. 11 is a buccal side view of the left wedge portion of the adjustable maxillary bite plane appliance of FIG. 6 where the wedge is in an expanded position;

FIG. 12 is a perspective view of a mandibular adjustable bite plane appliance base;

FIG. 13 is a perspective view of a mandibular adjustable bite plane appliance shim;

FIG. 14 is a cross-sectional view of the mandibular adjustable bite plane appliance base of FIG. 12; and

FIG. 15 is a cross-sectional view of the mandibular adjustable bite plane appliance shim of FIG. 13.

### DETAILED DESCRIPTION OF THE INVENTION

This application relates to an adjustable oral bite plane apparatus for applying to a patient's teeth.

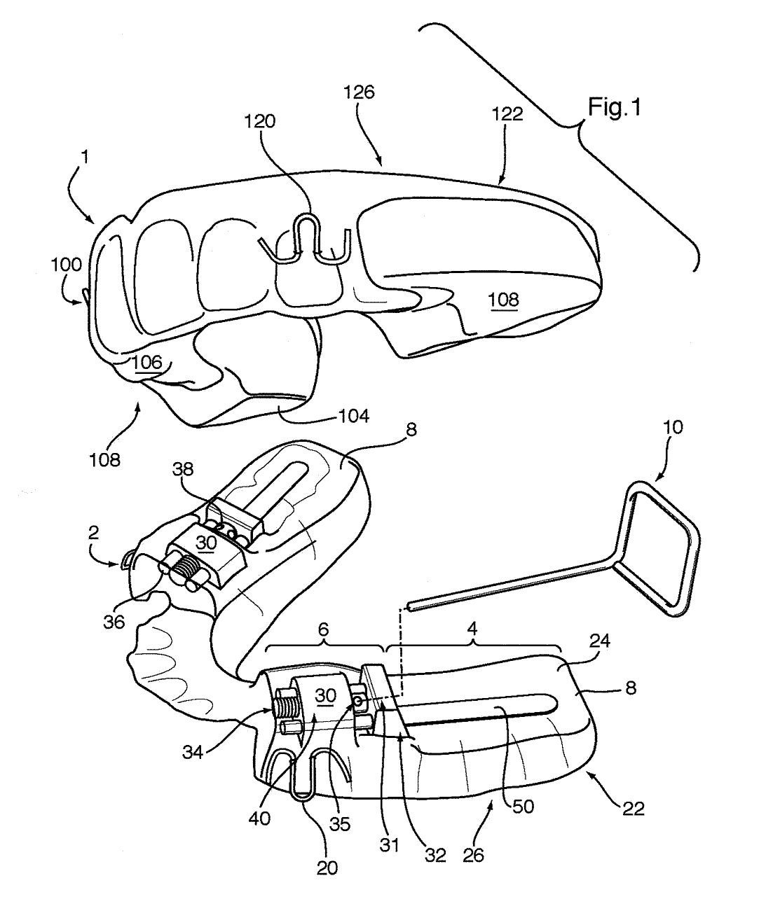

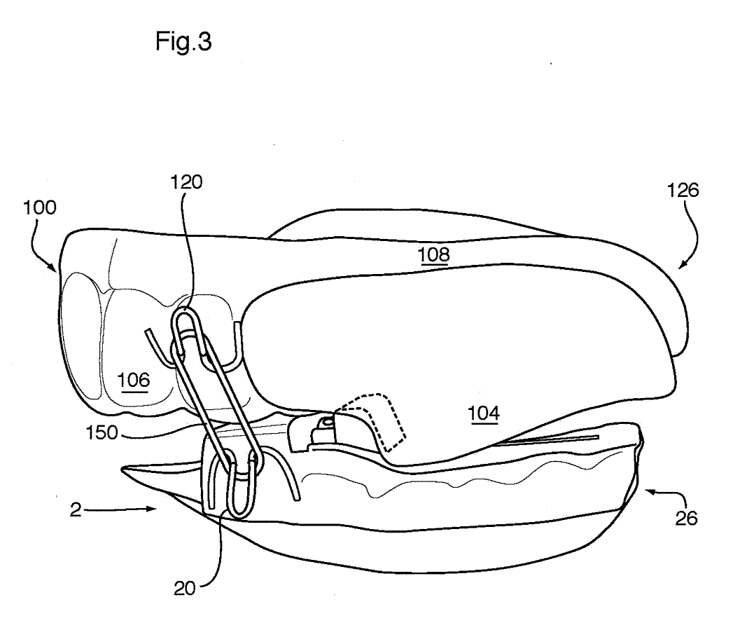

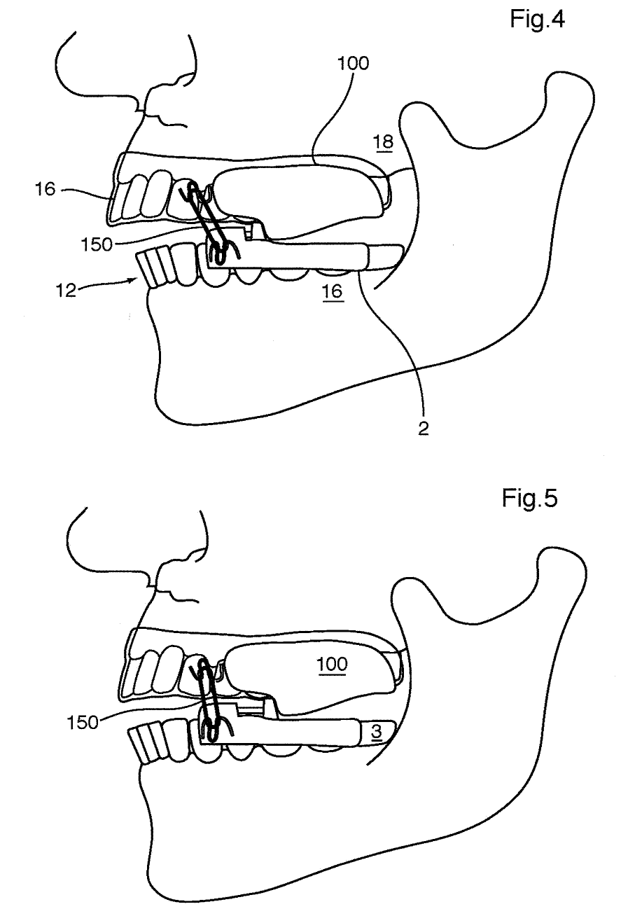

FIGS. 1 and 3 to 5 illustrate an adjustable oral bite plane apparatus 1 for disposing a patient's mandible anteriorly in accordance with one embodiment of the invention. The bite plane apparatus 1 comprises an adjustable mandibular bite plane appliance 2 and a maxillary bite plane appliance 100, each having a respective bite plane base 26, 126. The base 26, 126 may be formed from a rigid plastic such as Eclipse™ acrylic or Proform™, or a combination of rigid plastic and metal, which is suitable for the environment inside the mouth, molded on casts in a laboratory to generally conform to the patient's maxillary or mandibular dentition 12, 14 and soft tissues 16, 18 such that it is securely and removably held in place when mounted. The inner surfaces 22 of each base 26, 126 engages the patient's dentition 12, 14 and soft tissues 16, 18 when worn.

The base 26 of the adjustable mandibular bite plane appliance 2 has an inner surface 22 that conforms to at least a portion of a patient's mandibular dentition 12 while the outer surface 24 is exposed to the oral cavity when worn. The mandibular bite plane appliance 2 has at least one occlusal portion 8. For example, the mandibular bite plane appliance 2 may comprise left and right posterior occlusal portions 8 that are substantially mirror images of one another and that have inner surfaces that conform to the patient's molars. Each occlusal portion 8 has a raised section 6 and a recessed occlusal section 4. That is, the raised section 6 has a greater occlusal thickness than the occlusal section 4.

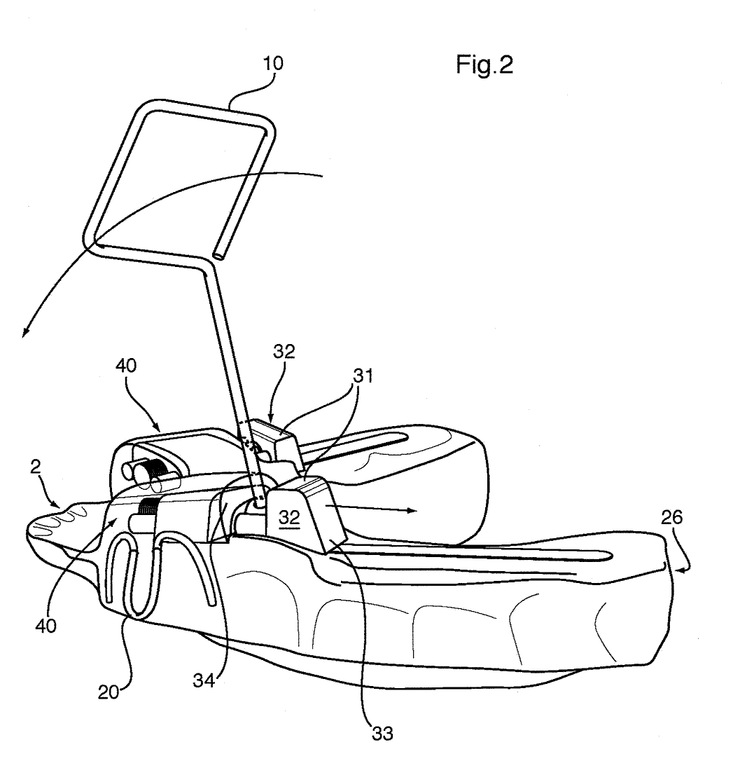

The raised section comprises a fixed portion 40 and a movable member 32. The fixed portion 40 may be integrally formed with the base 26. In the example of FIGS. 1 to 5 the movable member 32 is a block that is movable along the outer surface of the occlusal section 4. The movable block has a transverse width that is substantially the same as or slightly shorter than the transverse width of the fixed portion 40. The depth of the movable block may be substantially the same as the height of the outer occlusal surface of the fixed portion 40 from the outer occlusal surface of the occlusal section 4 such that the occlusal surface 31 of the movable block is substantially in the same plane as the outer occlusal surface of the fixed portion 40. As illustrated in FIGS. 1 to 5, the movable block may comprise a sloped surface 33 that defines the movable boundary between the raised section 6 and the occlusal section 4. Alternatively, the surface 33 may be vertical surface. The movable block may be made of a rigid and durable material such as a biocompatible metal.

The movable member 32 is joined to the fixed portion 40 of the raised section 6 by an expansion fastener 35 such as an expansion screw. The expansion fastener 35 may be adjusted to cause the movable member 32 to be advanced away from the fixed portion 40 and into the occlusal section, thereby lengthening the raised section 6 and shortening the occlusal section 4. The expansion fastener 35 may also be adjusted to cause the movable member to be retracted toward the fixed portion 40 such that the raised section 6 is shortened and the occlusal section 4 is lengthened.

The expansion fastener 35 may comprise a shaft 34 with a head end rotatably secured to the movable member 32 such that the shaft 34 is rotatable about a longitudinal axis of the shaft while the orientation of the movable member 32 remains the same in relation to the fixed portion 40, a threaded portion for engaging the fixed portion 40 and means for axial rotation of the shaft 34. In this embodiment, the fixed portion 40 comprises a complementary threaded receptacle for receiving the threaded portion of the shaft.

In the embodiment shown in FIGS. 1 to 5, the fixed portion 40 comprises an embedded block 30 and the complementary threaded receptacle is a complementary threaded hole in the embedded block 30. The embedded block 30 may be made of biocompatible metal and may have a front or shaft receiving surface 42 exposed to the oral cavity. The shaft 34 may comprise a section between the head end and the threaded portion with one or more transverse passageways 38 for receiving a key 10 to facilitate axial rotation of the shaft 34. In this embodiment, the expansion fastener 35 is adjusted to advance the movable member 32 away from the fixed section 40 by inserting the key 10 into one of the transverse passageways 38 and using the key 10 to rotate the shaft 34 in one direction. In a similar manner, the expansion fastener 35 may also be adjusted to retract the movable member 32 toward the fixed section 40 by rotating the shaft 34 in the opposite direction.

Movement of the movable member 32 may be guided by one or more guide rails 36, each guide rail 36 extending substantially parallel to the shaft 34 from the movable member 32 into a rail receptacle in the fixed portion 40. Where the fixed portion comprises an embedded block 30, the rail receptacle may be an aperture through or receptacle in the embedded block 30. The guide rails 36 may be integrally formed with the movable block 32. In the embodiment illustrated in FIGS. 1 to 5, two guide rails 36 extend from the movable block 32 into corresponding holes through the embedded block 30. The shaft 34 and guide rails 36 may be of such length that they extend through embedded block 30 when the expansion fastener 35 is in a contracted or partially contracted configuration. The fixed portion 40 may comprise one or more cavities extending from the rear surface 44 of the embedded block 30 to accommodate the portions of the shaft 34 and guide rails 36 that may protrude from the rear surface 44 of the embedded block 30.

The movement of the movable member 32 may be further guided by a track 50 extending longitudinally from the raised section 32 along the outer surface of the occlusal section 4. The track 50 may also retain the movable member 32 against the outer surface of the occlusal section. Where the movable member 32 is a block made of metal or some other hard material and the outer surface of the occlusal section 4 is made of plastic or some other material softer than the block, the track 50 may also prevent abrasion of the outer surface of the occlusal section 4 by the block. The track 50 may comprise a track member, such as a metal strip, that extends from the fixed section and is slidably engaged by the movable member 32.

The maxillary bite plane appliance 100 has a bite plane base 126, an inner surface 122 of which engages the patient's dentition 16 and soft tissue 18. The maxillary bite plane appliance 100 has at least one occlusal portion 108. The maxillary bite plane appliance 100 may comprise left and right posterior oclusal portions 108 that are substantially mirror images of one another and that have inner surfaces that conform to the patient's molars. Each occlusal portion 108 has a recessed section 106 and a raised section 104. That is, the raise section 104 has a greater occlusal thickness than the occlusal section 106.

As shown in FIGS. 1 to 5, the adjustable oral bite plane apparatus 1 may be used advantageously in a posterior bite plane appliance to dispose the patient's jaw anteriorly to prevent snoring. In this embodiment, the adjustable mandibular bite plane appliance 2 and maxillary bite plane appliance 100 are optionally provided with one or more tensioning pins, pegs or hooks 20, 120 to which tensioning members 150, such as an elastic band, may be attached to pull the patient's jaws closed and the mandible anteriorly. The mandibular tensioning hooks 20 are advantageously located on the buccal surfaces of the fixed portion 40 of the posterior occlusal portions 8 of the adjustable mandibular bite plane appliance 2 and the maxillary hooks 120 are advantageously located on the anterior buccal or labial surfaces of the maxillary bite plane appliance 100 such that the tension in the elastic bands 150 draws the lower jaw in an anterior direction. At least one optional elastic band 150 connects the left occlusal portion 8 of the mandibular adjustable bite plane appliance 2 to the left side of the maxillary bite plane appliance. Likewise, at least one elastic band 150 connects the right occlusal portion 8 of the mandibular adjustable bite plane appliance 2 to the right side of the maxillary bite plane appliance.

When the jaws of the patient are closed, the mandibular and maxillary bite plane appliances engage with each other. Particularly, the raised section 6 of the mandibular appliance engages with the recessed section 106 of the maxillary bite plane 100 and the recessed section 4 of the mandibular appliance engages with the raised section 104 of the maxillary bite plane. In this way, the mandibular bite plane, is restricted from posterior movement and the surface 33 of the movable member 32 when engaged with the maxillary bite plane appliance forces the lower jaw in an anterior direction.

In an embodiment the left and right posterior occlusal portions of the maxillary bite plane appliance 100 may have outer occlusal surfaces shaped to dispose the lower jaw anteriorly. Preferably, the mandibular bite plane has a raised anterior occlusal portion 6 and the maxillary bite plane has a recessed anterior occlusal portion 106 for abutting the corresponding raised anterior occlusal portion 6. When the removable bite planes 2, 100 are brought together by biting, they will not be properly seated unless the recessed anterior portion 106 of the maxillary bite plane abut the raised anterior portion 6 thereby disposing the lower jaw anteriorly. In this embodiment the maxillary bite plane and/or the mandibular bite plane may further comprise a chamfered protrusion which serves as a cam, abutting the posterior end 104 and 6 as the jaw is closed to force the mandible anteriorly and to further assist in guiding the lower jaw to the desired anterior disposition.

A removable bite plane appliance 1 according to this embodiment thereby allows adjustments with three degrees of freedom: along the vertical axis by making the raised portions 6, 104 of differing thicknesses; along the anterior-posterior axis by the shape of the outer occlusal surface of the bite planes and adjusting the movable member 32 and tension provided by the tensioning members 150; and, laterally by the elasticity and flexibility of the elastic bands 150. The lateral flexibility in particular allows patients who grind their teeth when sleeping to continue motion in that direction, thereby preventing or alleviating soreness in the jaw that is known to result when their lateral jaw motion is restricted.

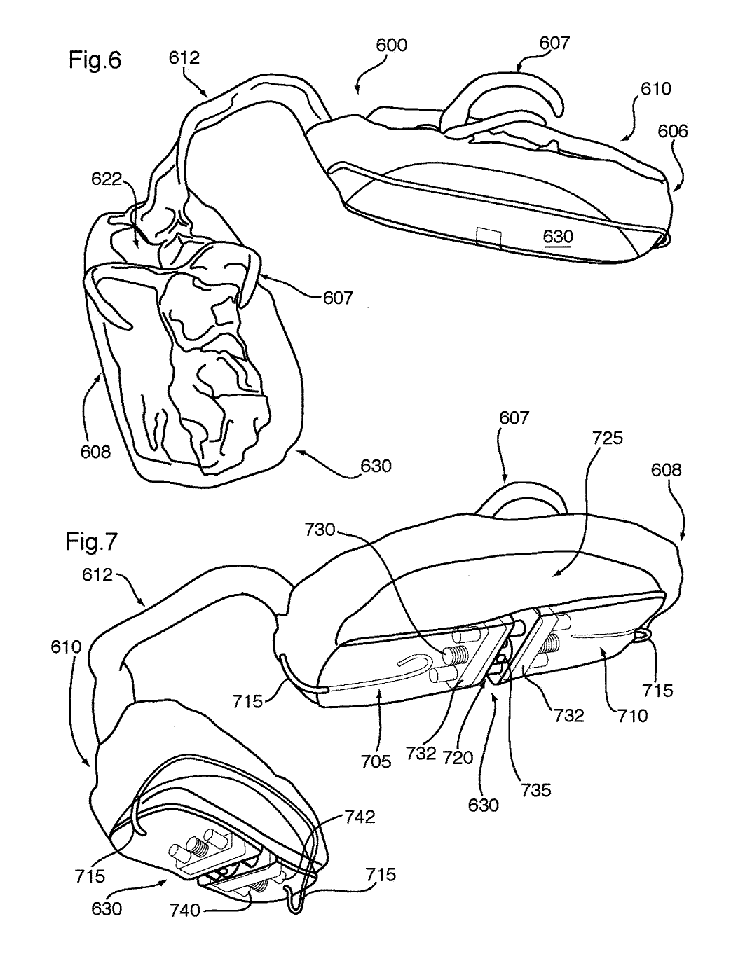

In a further embodiment, the bite plane (either the maxillary or mandibular) contains adjustable wedges. FIGS. 6 and 7 show an adjustable maxillary bite plane in an embodiment of the invention. The bite plane 600 contains a bite plane base 606. The bite plane base may be formed from a rigid plastic such as Eclipse acrylic or Proform, or a combination of rigid plastic and metal, which is suitable for the environment inside the mouth, molded on casts in a laboratory to generally conform to the patient's maxillary or mandibular dentition 12, 14 and soft tissue 16, 18 such that it is securely and removably held in place when mounted. The inner surface 622 of the base 606 engages the patient's dentition 12, 14 and soft tissue 16, 18 when worn. The base 606 may include clips 607 that engage the patient's molars. The clips 607 may be made from metal material that while malleable to allow the bite plane to be inserted, holds its shape to securely hold the bite plane in place.

The bite plane 600 has a left and right occlusal portions 608 and 610 corresponding to the right and left sides of the mandibular or maxillary. The left and right occlusal portions are connected using a bridge 612.

The bridge 612 may be made from the same material as the bite plane and serves to locate and retain the occlusal portions in alignment with the patient's maxillary or mandibular dentition. The bridge 612 also serves to retain the occlusal portions when the bite plane is being inserted into the patient's mouth or if the bite plane becomes detached from the mandibular or maxillary. Since the structure formed by the two occlusal portions joined by the bridge 612 is larger than either of the occlusal portions alone, the structure is less likely to be accidentally swallowed by the patient or lost.

The adjustable wedges 630 formed in each occlusal portion 608, 610 generally allow the occlusal surface of the bite plane to be raised or lowered relative to the occlusal surface of the patient's teeth.

In a preferred embodiment, the adjustable wedges may be formed within the occlusal portions of the bite plane. The occlusal base portion 606 preferably has a depression 725 opening away from the patient's teeth. The depression may be sloped or curved away from the deepest part of the depression in the middle of the occlusal portion.

With reference to FIG. 7, a first and second wedge parts 705 and 710 for the adjustable wedge with the occlusal surfaces of the first and second wedge parts for the occlusal surface of the occlusal portion, are retained in slidable contact with the occlusal portion in the depression 725 by a retaining member 715. The retaining member may be a metal wire embedded in the plastic of the wedge part and the occlusal portion. The wedges 705, 710 are adjustably connected to each other by an expansion fastener 720. The expansion fastener 720 may be adjusted to cause the wedge parts to be closer or farther apart from each other.

With reference to FIGS. 9 and 11, as the first and second wedge parts are caused to be farther apart, the wedge parts are forced against the retainer member 715 and slide along the sloped portion of the depression 725. As the wedge parts slide along the sloped portion of the depression 725, the distance from the occlusal surface of the wedge to the patient's teeth is increased.

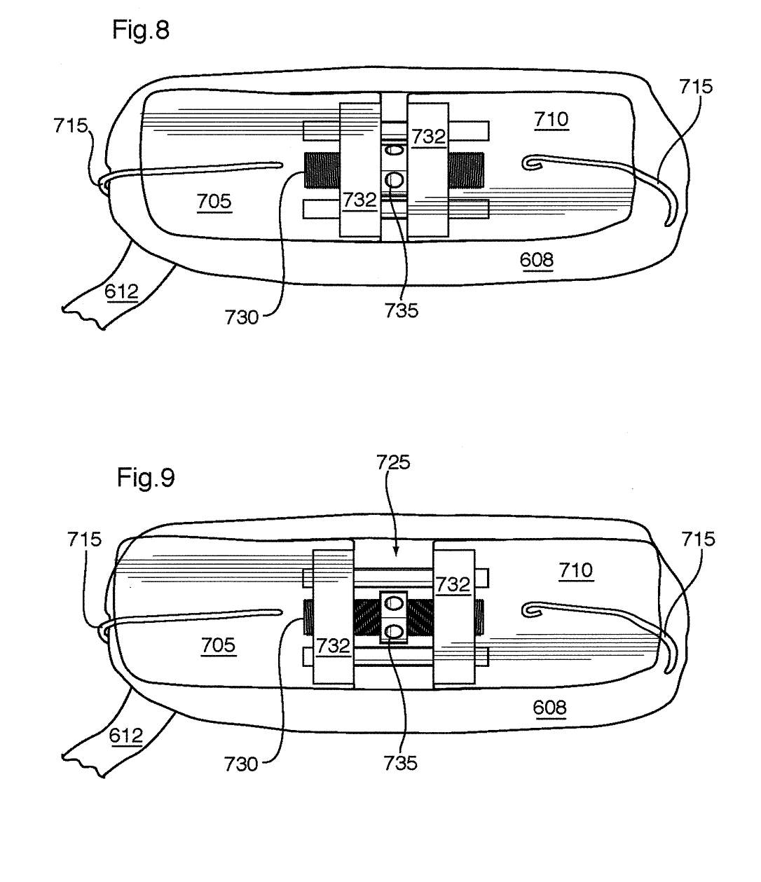

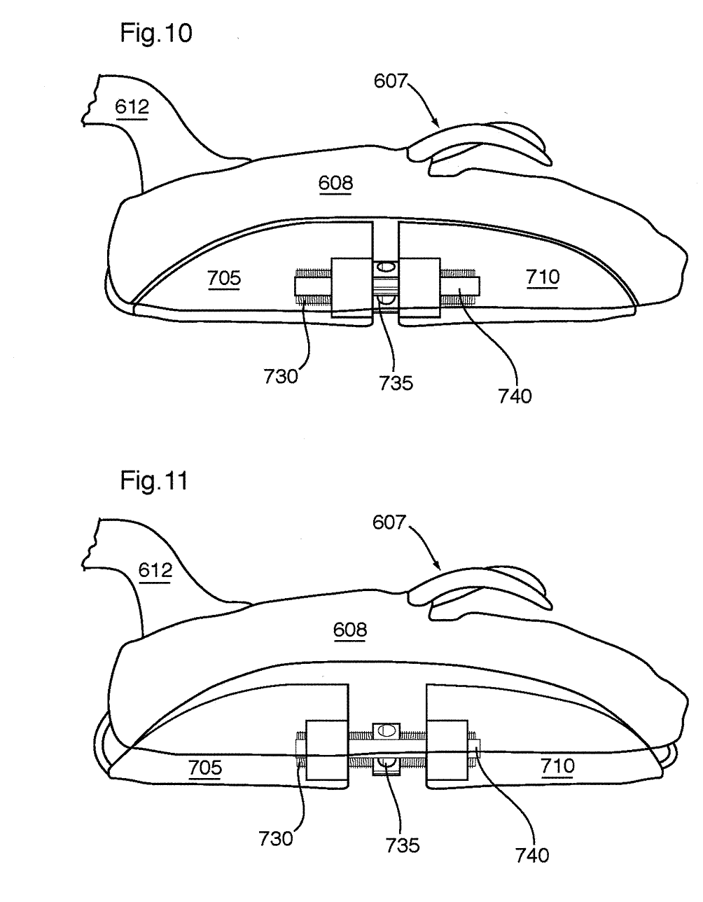

With reference to FIGS. 8 and 10, conversely, as the wedge parts are caused to be closer together, the retainer member 715 forces the wedge parts into the depression 725 as the wedge parts slide along the slopped surface. As a result, the distance between the occlusal surface of the wedge and the occlusal surface of the patient's teeth is reduced.

The expansion fastener 720 may comprise an expansion screw 730 rotatably secure within the first and second wedge parts 705 and 710. Threads on the expansion screw preferably engage one or both of the wedge parts using complementary threaded receptacles 732 for receiving the expansion screw so that rotations of the expansion screw cause the wedge parts to be forced further apart or drawn closer together. The two threaded receptacles 732 may be embedded within the first and second wedge parts.

The expansion screw 730 may contain a nut 735 comprising one or more openings, either partial or fully penetrating the nut 735. These openings, preferably circular holes through the nut are capable of receiving a key 10 for rotating the expansion screw 730 by a dentist or other operator. The key 10 may be similar to the key 10 previously described.

The movement of the adjustable wedge parts may be constrained by one or more pins 740, 742 aligning the first and second wedge parts. In a preferred embodiment, there are two pins. A first pin 740 may be secured in the first wedge part 705 and slideably restrained in the second wedge part 710. A second pin 742 may be secured in the second wedge part 710 and slideably restrained in the first wedge part 705. In this way, the distance between the first and second wedge parts may be adjusted using the expansion screw while the occlusal surfaces of the first and second wedge parts remain in alignment.

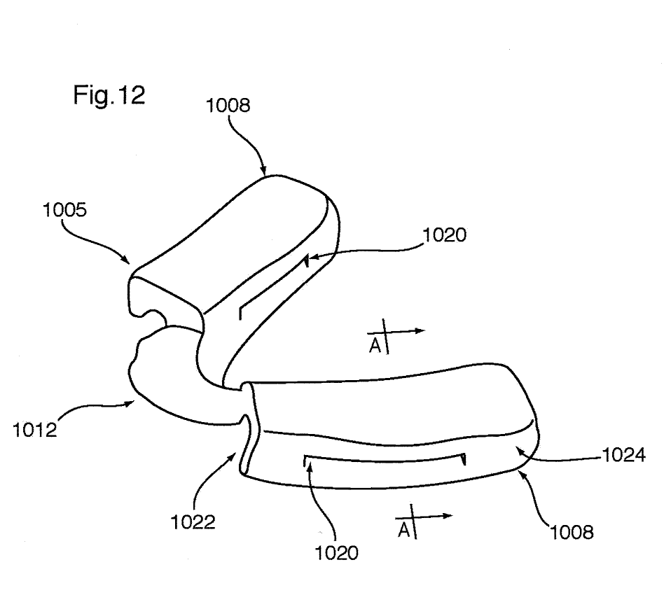

With reference to FIG. 12 in an embodiment of the invention includes an adjustable oral bite plane apparatus 1000. The bite plane apparatus comprises an adjustable bite base 1005 that may be formed to engage a patients mandibular or maxillar dentition and surrounding soft tissue.

The bite plane has two occlusal portions 1008 that are substantially mirror images of one another. Each occlusal portion has an inner surface 1022 that conform to the patient's molars. The bite plane may be formed to fit a specific patient's molars using a mold made from a cast of the teeth. The two occlusal portions 1008 are joined by a bridge 1012. Each occlusal portion 1008 has an outer surface that faces the patient's mouth.

Each of the outer surfaces of the occlusal portions of the bite plane base have one or more retainers 1020, preferably formed within the occlusal portion 1008. These retainers mate with the removable shims described below. In a preferred embodiment, the retainers are indentations formed in the base portion compatible with detents in the adjustable shims

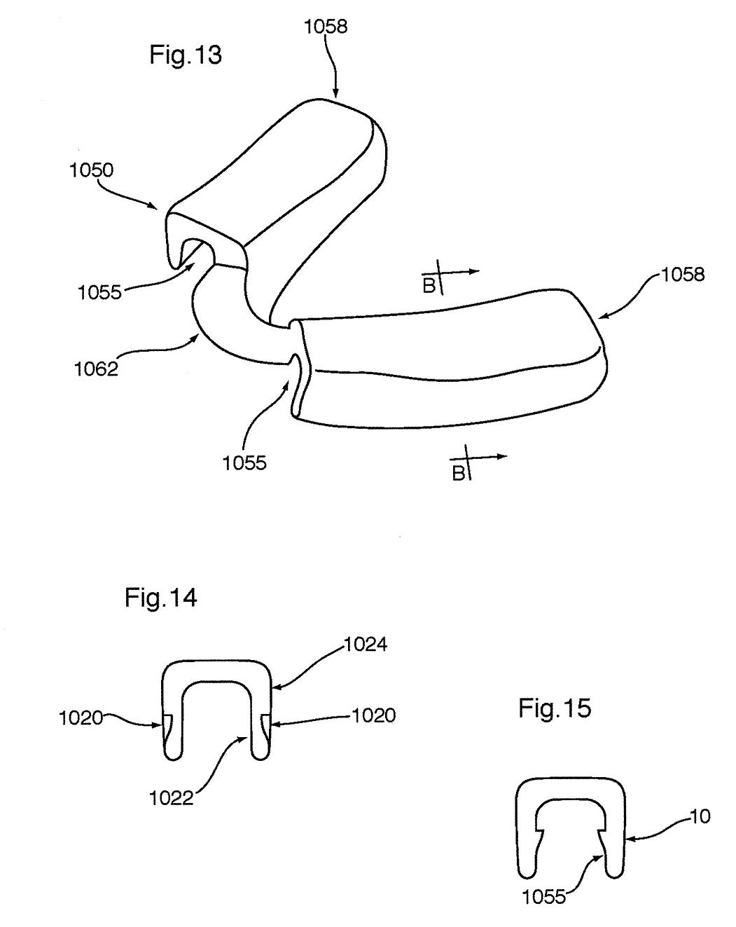

An adjustable shim 1050, with reference to FIG. 13, preferably is compatible with the occlusal base 1005. A shim comprises two occlusal portions 1058 and a bridge portion 1062 connecting the two occlusal portions 1058. The occlusal portions each have an inner surface 1055 which is complementary to the outer surface 1024 of the occlusal portion of the base. The occlusal portions 1058 of the adjustable shim has detents 1060, preferably molded into the inner surface 1055 of the occlusal portion that engages the retainers 1020 on the occlusal portion 1008 of the base 1005. In this way, the adjustable shim can be placed over the base and be retained in place by the retainers.

The adjustable shims may be made of a variety of thicknesses. The thickness of the material between the inner surface and outer surface of the occlusal portion, in combination with the thickness of the base portion of the base determines the spacing between the mandibular and maxillary when the patient closes their mouth.

By having the adjustable shims releasably retained against the base, the adjustable shim can be removed from the base, and a different adjustable shim retained against the base. In this way the thickness of the bite plate can be changed without requiring a new bite plate base to be made.

As mentioned above, a bite plate base conforms to the patients molars, whereas the adjustable shims are complementary with the outer surface of the bite plane base.

Various embodiments of the present invention having been thus described in detail by way of example, it will be apparent to those skilled in the art that variations and modifications may be made without departing from the invention. The invention includes all such variations and modifications as fall within the scope of the appended claims.

A portion of the disclosure of this patent document contains material which is subject to copyright protection. The copyright owner has no objection to the facsimile reproduction by any one of the patent document or patent disclosure, as it appears in the Patent and Trademark Office patent file or records, but otherwise reserves all copyrights whatsoever.

What is claimed is:

1. An adjustable oral bite plane appliance for mounting over at least a portion of a patient's dentition, the bite plane appliance comprising at least one occlusal portion comprising a raised section and an occlusal section, the raised section comprising a fixed portion and a movable member for extending the raised section into the occlusal section, the movable member joined to a fixed portion of the raised section by an expansion fastener, whereby expansion of the fastener causes the movable member to be advanced into the occlusal section.

2. The bite plane appliance of claim 1 wherein the expansion fastener comprises a pin comprising:
a head end rotatably secured to the movable member;
a threaded portion for engaging the fixed portion; and
means for axial rotation of the pin;
wherein the fixed portion comprises a complementary threaded receptacle for engaging the threaded portion of the pin and wherein rotating the pin in one direction causes the movable member to be advanced into the occlusal section.

3. The bite plane appliance of claim 2 further comprising at least one guide rail extending from the movable member into at least one corresponding rail receptacle in the fixed portion, each rail receptacle guiding longitudinal movement of the corresponding guide rail and preventing lateral movement of the corresponding guide rail.

4. The bite plane appliance of claim 1 wherein the expansion fastener comprises an expansion screw.

5. The bite plane appliance of claim 2 wherein the occlusal section comprises a track that extends longitudinally from the fixed portion for guiding the movement of the movable member.

6. The bite plane appliance of claim 5 wherein the track comprises a track member that extends longitudinally from the fixed portion along the occlusal surface of the recessed portion and wherein the movable member slidably engages the track member.

7. The bite plane appliance of claim 1 comprising a left posterior occlusal portion and a right posterior occlusal portion.

8. The bite plane appliance of claim 1 wherein the raised section is anterior to the occlusal section.

9. The bite plane appliance of claim 1 further comprising at least one tensioning pin protruding from a buccal surface of the raised section for attachment of a tensioning member for connection with a complementary bite plane appliance to draw the patient's jaws to a desired position.

10. An adjustable oral bite plane appliance comprising:
a base for mounting over a patient's teeth, the base comprising at least one occlusal portion having an outer occlusal surface comprising a depression for retaining an expandable wedge, the depression being deepest toward the centre of the depression and tapering toward an anterior end along an anterior wall of the depression and tapering toward a posterior end along a posterior wall of the depression;
the expandable wedge seated in each depression comprising an anterior wedge portion, a posterior wedge portion and a gap toward the centre of the depression along the longitudinal axis of the occlusal portion between the anterior wedge portion and the posterior wedge portion, wherein:
each wedge portion has a thickness that is thickest at the gap;
each wedge portion tapers toward the anterior and posterior ends of the depression;
the anterior wedge portion is connected to the posterior wedge portion by an expansion fastener comprising adjustment means accessible via the gap; and
each wedge portion is connected to the base by at least one flexible member for retaining at least a part of the wedge portion within the depression, wherein the at least one flexible member comprises a wedge portion end connected to the wedge portion and a base end connected to the base;
wherein each occlusal portion-wedge pair defines an adjustable portion of the bite plane appliance and whereby the occlusal thickness of the adjustable portion is increased by adjusting the expansion fastener to expand the expandable wedge to cause the expandable wedge to ride up the anterior and posterior walls of the depression and the occlusal thickness of the adjustable portion is decreased by adjusting the expansion fastener to contract the expandable wedge to allow the expandable wedge to slide down the anterior and posterior walls and into the depression.

11. The bite plane appliance of claim 10 wherein the expansion fastener comprises a pin comprising:
a head end secured to one of the anterior wedge portion or the posterior wedge portion such that the pin is rotatable about a longitudinal axis of the pin while the orientation of the anterior wedge portion remains the same in relation to the posterior wedge portion;
a threaded portion for engaging the other of the anterior wedge portion or the posterior wedge portion; and
means for rotating the pin;
wherein the other of the anterior wedge portion or the posterior wedge portion comprises a complementary threaded receptacle for engaging the threaded portion of the pin.

12. The bite plane appliance of claim 10 wherein the expansion fastener comprises a pin comprising:
an anterior threaded portion toward an anterior end of the pin;
a posterior threaded portion toward a posterior end of the pin; and
wherein adjustment means comprises at least one transectional receptacle toward the longitudinal centre of the pin for receiving a key for rotating the pin about the longitudinal axis of the pin; and
wherein the anterior wedge portion comprises a first complementary threaded receptacle for engaging the anterior threaded portion of the pin and the posterior portion comprises a second complementary threaded receptacle for engaging the posterior threaded portion of the pin.

13. The bite plane appliance of claim 10 further comprising at least one guide rail, each guide rail extending from one of the anterior wedge portion or the posterior wedge portion into at least one corresponding receptacle in the other of the anterior wedge portion or the posterior wedge portion, each corresponding receptacle guiding longitudinal movement of the corresponding guide rail and preventing lateral movement of the corresponding guide rail.

14. The bite plane appliance of claim 10 wherein the expansion fastener comprises an expansion screw.

15. An adjustable oral bite plane apparatus for disposing a patient's mandible anteriorly, the apparatus comprising:
an adjustable bite plane appliance of claim 9 wherein the adjustable bite plane appliance is for mounting over at least a portion of the patient's mandibular dentition and comprises a left posterior occlusal portion and a right posterior occlusal portion, wherein the raised section of each portion is anterior to the occlusal section of the same portion; and
a maxillary bite plane appliance for mounting over at least a portion of the patient's maxillary dentition, the maxillary bite plane appliance comprising a left posterior occlusal portion, a right posterior occlusal portion and at least one tensioning pin protruding from a buccal surface of the maxillary bite plane appliance for attachment of the at least one tensioning member from the adjustable bite plane appliance, each occlusal portion comprising a raised portion for abutting the occlusal section of the adjustable bite plane appliance.

16. The adjustable oral bite plane apparatus of claim 15 wherein the maxillary bite plane appliance comprises:
a base substantially conforming to at least a portion of the patient's maxillary dentition, the maxillary base comprising an inner surface that engages at least a portion of the patient's maxillary dentition, an outer surface, a left posterior occlusal portion and a right posterior occlusal portion; and
left and right maxillary removable occlusal shims, each occlusal shim being attachable to the posterior portion of the outer surface of the base and comprising:
an inner surface that substantially conforms to the outer surface of at least a portion of one of the posterior occlusal portions of the base; and
a raised occlusal portion.
